(19) United States
(12) Patent Application Publication
(10) Pub. No.: US 2015/0216688 A1
(43) Pub. Date: Aug. 6, 2015

(54) Intravascular Medical Device Release System

(71) Applicant: The Texas A&M University System, College Station, TX (US)

(72) Inventors: Todd Lawrence Landsman, College Station, TX (US); Wonjun Hwang, College Station, TX (US)

(21) Appl. No.: 14/612,631

(22) Filed: Feb. 3, 2015

Related U.S. Application Data
(60) Provisional application No. 61/935,639, filed on Feb. 4, 2014.

(51) Int. Cl. A61F 2/95

(52) U.S. Cl. CPC A61F 2/95; A61F 2002/9522

(57) ABSTRACT

The present invention provides a braided coil catheter to delivery of a vascular implant through a catheter including a braided coil adapted to at least partially retain a vascular implant comprising a braided coil distal tip, a braided coil proximal end connected by a braided coil body having a lumen with a lumen diameter; a friction plug comprising a proximal friction plug end, a distal friction plug end connected by a friction plug body with a friction plug diameter greater than the lumen diameter to frictionally fit into the lumen of the braided coil distal tip; a vascular implant having an implant mating end, an implant non-mating end connected by an implant body wherein the implant mating end is connected to the distal friction plug end; and a pusher catheter inserted into the lumen and to contact the proximal friction plug end and force the friction plug from the lumen.

### CROSS-REFERENCE TO RELATED APPLICATIONS

This application claims benefit of U.S. Provisional Application Ser. No. 61/935,639 entitled “Intravascular Medical Device Release System” which was filed on Feb. 4, 2014 and is incorporated herein by reference in its entirety.

### STATEMENT OF FEDERALLY FUNDED RESEARCH

This invention was made with government support under Grant No. R01EB000462 awarded by the National Institutes of Health (NIH). The government has certain rights in the invention.

### BACKGROUND OF THE INVENTION

Without limiting the scope of the invention, its background is described in connection with medical devices and more specifically, to an apparatus that can deliver various vascular implants via a catheter into different regions of the human vasculature.

U.S. Pat. No. 8,579,988 discloses a medical device delivery catheter to a location in a patient's body includes an elongate catheter body having a proximal end and a distal end, a pod coupled with the distal end of the catheter body and adapted to house the medical device during delivery to the location and to open to release the medical device, and at least one distal actuator coupled with at least one of the pod and the medical device. The distal actuator is adapted to promote opening of the pod. A method involves advancing a pod at the distal end of an elongate catheter to the location within the body and activating an actuator coupled with the pod and/or the medical device to cause the pod to open.

U.S. Pat. No. 7,901,396 discloses a transvenous medical device delivery system and method of manufacture that includes an outer catheter, a first inner catheter insertable through the outer catheter, and a second inner catheter insertable through the first inner catheter and adapted to receive a guide wire extending there through. The first inner catheter includes a braided reinforcement layer and is adapted to receive an elongated medical device extending there through.

U.S. Pat. No. 6,059,779 discloses a delivery catheter for electrolytically detachable implant for introducing a delivery member such as a guide wire having an implant coupled thereto via an electrolytically disintegratable link that is coupled to the anode of a power supply having an anode and a cathode.

### SUMMARY OF THE INVENTION

The present disclosure provides a universal system that can deliver a number of different vascular implants to various regions of the human vasculature reducing the need for specialized vascular implant delivery devices for specific regions of the body.

The present invention provides a braided coil catheter to deliver a vascular implant through a catheter including a braided coil adapted to at least partially retain a vascular implant comprising a braided coil distal tip, a braided coil proximal end connected by a braided coil body having a lumen with a lumen diameter; a friction plug comprising a proximal friction plug end, a distal friction plug end connected by a friction plug body with a friction plug diameter greater than the lumen diameter to frictionally fit into the lumen of the braided coil distal tip; a vascular implant having an implant mating end, an implant non-mating end connected by an implant body wherein the implant mating end is connected to the distal friction plug end; and a pusher catheter inserted into the lumen and to contact the proximal friction plug end wherein the pusher catheter can force the friction plug from the lumen.

The present invention also includes a method of implanting a vascular implant by moving a catheter from a vascular access site to an implant target site; providing an apparatus for delivery of a vascular implant comprising a braided coil comprising a braided coil distal end, a braided coil proximal end connected by a braided coil body having a lumen with a lumen diameter; a friction plug comprising a proximal friction plug end, a distal friction plug end connected by a friction plug body with a friction plug diameter greater than the lumen diameter frictionally fitted into the lumen of the braided coil distal end; providing a vascular implant having an implant mating end, an implant non-mating end connected by a an implant body wherein the implant mating end is connected to the proximal friction plug end; loading the vascular implant into the braided coil by frictionally fitting the friction plug into the braided coil to form a loaded braided coil; navigating the loaded braided coil through the catheter to reach the implant target site; advancing the push wire through the braided coil until the wire contacts the friction plug; ejecting the friction plug from the braided coil to deploy the vascular implant into the implant target site; and removing the braided coil and push wire from the catheter.

### BRIEF DESCRIPTION OF THE DRAWINGS

For a more complete understanding of the features and advantages of the present invention, reference is now made to the detailed description of the invention along with the accompanying figures and in which:

FIG. 1 shows a cross-section of the braided coil of one embodiment of the present invention.

FIG. 2 is a cross-sectional view of one embodiment of the braided coil of the present invention.

FIG. 3 is a cross-sectional view of one embodiment of braided coil and push wire of the present invention.

FIG. 4 is a cross-sectional view of one embodiment of the present invention that shows the friction plug being loaded into a braided coil.

FIG. 5 is a cross-sectional view of one embodiment of the present invention that shows the friction plug loaded into a braided coil and including push wire.

FIG. 6 is a cross-sectional view of one embodiment of the present invention that shows the delivery apparatus releasing the vascular implant.

FIG. 7 is a cross-sectional view of another embodiment of the instant invention that shows the friction plug loaded into a braided coil, including push wire and pull wire.

FIG. 8 is a cross-sectional view of another embodiment of the instant invention that shows the friction plug loaded into a braided coil, including push and pull wires.

FIG. 9 is a cross-sectional view of another embodiment of the instant invention that shows the friction plug 20 loaded into a braided coil and including pull wire.

FIG. 10 is a plot that shows the grip strength based on the diameter of the friction plug and the material of the braided coil.

FIG. 11 is a plot that shows the push force required to eject the friction plug from the braided coil as a function of the depth of the nitinol friction plug from the tip of the braided coil.

FIG. 12 is a graph that shows a comparison between a vascular implant delivery system of the instant invention and a guglielmi detachable coil detachment system.

FIG. 13 is a graph that shows the grip strength of a stainless steel braided coil as a function of the friction plug diameter.

FIG. 14 is another graph that shows the grip strength of a stainless steel braided coil.

FIG. 15 is a graph that shows the grip strength based on filar deflection on the braided coil.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The delivery apparatus provides a universal system that can be used to deliver a number of different implants and devices to various regions of the human vasculature. The delivery system described requires no power source and can be scaled to various sizes to be used in different regions of the human vasculature. Most current conventional implant delivery systems can only be used with the specific device the delivery system was designed for. The invention described herein has the potential to be a universal delivery system. Furthermore, the delivery system described is easily sterilized, extremely cheap and fast to manufacture. The lack of a power source allows this invention to be produced at a fraction of the cost of other typical delivery systems. This delivery apparatus also allows easy navigation through the neurovasculature, and provides a safe, effective means of delivering embolic devices to cerebral aneurysms, effectively reducing the risk of hemorrhagic stroke as a result of the aneurysm rupturing.

For example, the present invention provides a universal delivery system that includes a braided coil catheter for delivery of a vascular implant through a catheter. As used herein, the terms “braided coil” “braided shaft”, “coiled catheter” or “braided apparatus” refer to a braided or coiled shaft that provides a flexible shaft often used in devices used for minimally invasive surgeries. The pitch, thickness and materials used to make the braided coil can be varied depending on the usage and materials, as outlined in greater detail herein below. Factors that can be used to determine the materials, pitch, thickness of the braided coil include, but are not limited to: size, shape, torque, burst pressure resistance, pushability, steerability, and kink resistance. The catheter has a diameter that is greater than the diameter of the braided coil catheter, so that the braided coil catheter retains free movement in the catheter. The braided coil catheter includes a braided coil distal tip, a braided coil proximal end connected by a braided coil body with a lumen formed in the braided coil catheter with a lumen diameter. The lumen is adapted to at least partially retain a vascular implant. The vascular implant is connected to a friction plug to frictionally fit the friction plug and the implant in the lumen of the braided coil catheter. The friction plug includes a proximal friction plug end, a distal friction plug end connected by a friction plug body with a friction plug diameter greater than the lumen diameter to frictionally fit into the lumen of the braided coil distal tip. The vascular implant includes an implant mating end, an implant non-mating end connected by an implant body wherein the implant mating end is connected to the distal friction plug end. The friction plug and the implant are retained in the lumen to the point of where the implant is to be deployed. A pusher catheter is inserted in a freely advancing and retracting manner into the lumen and extended to contact the proximal friction plug end. The pusher catheter can then be pushed against the friction plug to overcome the frictional fitting of the friction plug in the lumen and expel the friction plug and the implant. As a result, it is necessary for the pusher catheter to be longer than the braided coil catheter and the catheter and retain flexibility so that it can pass through the lumen and structure to forcibly expel the friction plug from the lumen. It is understood that the optimal parameters (e.g., length and diameter) of the various components (e.g., catheter, braided coil catheter, pusher catheter and vascular implant) can be selected according to the disease condition and point of delivery.

In another embodiment, the present invention provides a universal delivery system that includes a braided coil catheter for delivery of a vascular implant through a catheter. The catheter has a diameter that is greater than the diameter of the braided coil catheter so that the braided coil catheter retains free movement in the catheter. The braided coil catheter includes a braided coil distal tip, a braided coil proximal end connected by a braided coil body with a lumen formed in the braided coil catheter with a lumen diameter. The lumen is adapted to at least partially retain a vascular implant. The vascular implant is connected to a friction plug to fit the friction plug and the implant in the lumen of the braided coil catheter. A retaining wire is positioned in the lumen between the friction plug and the inner wall of the braided coil catheter to frictionally retain the friction plug in the lumen. The retaining wire then extends through the braided coil catheter to exit the proximal end and allow removal of the retaining wire. The retaining wire is pulled to remove the retaining wire from between the friction plug and the inner wall of the braided coil catheter to release the friction plug from the braided coil catheter. The vascular implant includes an implant mating end, an implant non-mating end connected by an implant body wherein the implant mating end is connected to the distal friction plug end. The friction plug and the implant are retained in the lumen to the point of where the implant is to be deployed. A pusher catheter is inserted in a freely advancing and retracting manner into the lumen and extended to contact the proximal friction plug end. The pusher catheter can then be pushed against the friction plug to overcome the frictional fitting of the friction plug in the lumen and expel the friction plug and the implant. As a result, it is necessary for the pusher catheter to be longer than the braided coil catheter and the catheter to retain its flexibility so that it can pass through the lumen and structure to forcibly expel the friction plug from the lumen. It is understood that the optimal parameters (e.g., length and diameter) of the various components (e.g., catheter, braided coil catheter, pusher catheter and vascular implant) can be selected according to the disease condition and point of delivery. The retaining wire may be made from any materials known to the skilled artisan. For example, the retaining wire may be a long thin member formed of polymer material (e.g., polyamide resin) for example, a thread that forms the retaining wire and the strength may be increased by forming the retaining wire into a loop.

In another embodiment, the present invention provides a universal delivery system that includes a braided coil catheter for delivery of a vascular implant through a catheter. The catheter has a diameter that is greater than the diameter of the braided coil catheter so that the braided coil catheter retains free movement in the catheter. The braided coil catheter includes a braided coil distal tip, a braided coil proximal end connected by a braided coil body with a lumen formed in the braided coil catheter with a lumen diameter. The lumen is adapted to at least partially retain a vascular implant. The braided coil catheter includes a restriction in the braided coil distal tip to fit the friction plug and frictionally retain the friction plug. The restriction is a restriction of filar deflection via welding filars together, or placing a solid band around the braided coil to prevent filar deflection. In another embodiment the restriction may be an indention.

The vascular implant is connected to a friction plug to frictionally fit the friction plug and the implant in the lumen of the braided coil catheter. The friction plug includes a proximal friction plug end, a distal friction plug end connected by a friction plug body with a friction plug diameter greater than the lumen diameter to frictionally fit into the lumen of the braided coil distal tip. The vascular implant includes an implant mating end, an implant non-mating end connected by an implant body wherein the implant mating end is connected to the distal friction plug end. The friction plug and the implant are retained in the lumen to the point where the implant is to be deployed. A pusher catheter is inserted in a freely advancing and retracting manner into the lumen and extended to contact the proximal friction plug end. The pusher catheter can then be pushed against the friction plug to overcome the frictional fitting of the friction plug in the lumen and expel the friction plug and the implant. As a result, it is necessary for the pusher catheter to be longer than the braided coil catheter and the catheter will retain flexibility, so that it can pass through the lumen and structure to forcibly expel the friction plug from the lumen. It is understood, that the optimal parameters (e.g., length and diameter) of the various components (e.g., catheter, braided coil catheter, pusher catheter, and vascular implant) can be selected according to the disease condition and point of delivery.

In addition, the invention may include a marker for X-ray imaging. The X-ray imaging marker may be embedded in or wrapped around the braided coil distal tip, and then the device can be delivered while viewing the X-ray image.

The use of the friction force by the braided coil is novel in comparison to other delivery systems. This system has rarely been investigated because the frictional fit between two mating surfaces is rarely perceived as providing a strong enough grip force to deliver a medical device or implant. Most other inventions instead utilize miniaturized latches, hooks, pods, and electrolytically disintegrable links to provide a stronger grip on the implant.

One embodiment of the present invention is to use the delivery apparatus to implant a shape memory polymer (SMP) embolic foam device to treat vascular abnormalities comprising of cerebral aneurysm, intracranial aneurysms, arteriovenous malformation (AVM), arteriovenous fistula (AVF), peripheral vasculature, and neurovascular abnormalities. Thousands of individuals are diagnosed with vulnerable cerebral aneurysms every year. There are a variety of ways to treat aneurysms ranging from an extravascular approach, which involves intrusive surgery or microsurgery, to extra-intravascular and endovascular approaches, which are less invasive. There has been an emerging trend in modern medicine and surgery to develop devices and methods for performing minimally invasive procedures. The endovascular approach which is the least invasive approach involves using a catheter to access the interior of the aneurysm as shown in Engleson, U.S. Pat. No. 4,884,579 and incorporated herein by reference, to deliver an embolic device. The device redirects blood flow away from the aneurysm allowing a thrombus to form in the aneurysm. This prevents the aneurysm from rupturing and will potentially shrink in size over time.

In another embodiment of the present invention, the apparatus for delivery of a vascular implant via a catheter comprises of a braided coil having a mating end, a proximal end, and a lumen with a lumen diameter; a friction plug frictionally fitted into the mating end of the lumen wherein the friction plug has a distal end, a second end, and a plug diameter larger than the lumen diameter; a vascular implant having a mating end and a non-mating end wherein the mating end of the vascular implant is connected to the distal end of the friction plug; a push wire insertable into the braided coil wherein the push wire has a wire diameter smaller than the lumen diameter; and a handle connectable to the push wire, the braided coil, or both wherein the handle can advance or retract the push wire within the braided coil.

In one aspect of the apparatus for delivery of a vascular implant via a catheter, a braided coil is used to grip the vascular implant and transport it via a catheter to the implant site. In another aspect, the braided coil has a lumen, which the friction plug goes into. In different embodiments of the invention, the lumen can have a diameter of 0.005, 0.0055, 0.006, 0.0065 or 0.007 inches. In another aspect, the outer diameter of the braided coil can be 0.01, 0.011, 0.012, 0.013, 0.014, 0.015, 0.016, 0.017, 0.018 inches. The friction plug and vascular implant loaded into the braided coil needs to be able to fit inside a catheter and navigate within it freely. In another aspect, the braided coil is also comprised of a set of filars that when not fixed together at a terminating tip, are free to deflect and expand the diameter of the braided coil to a mating diameter larger than the lumen diameter. The deflection of the set of filars is what allows the friction plug to be press fitted into the braided coil. The filars of the braided coil will distend and apply a spring force to grip the friction plug during transport. The braided coil comprises of nitinol, nickel titanium, titanium alloys, brass, nickel aluminum, bronze, iron alloys, stainless steel, platinum, gold, tantalum, titanium, tungsten, metals, psuedoelastic metal alloys, shape memory alloys, polyurethane, polytetrafluoroethylene, polyvinyl alcohol, polyester, silicone, or acrylic. In yet another aspect of the present invention, the braided coil also has pre-formed grooves to grip the friction plug.

In another aspect, the friction plug is shaped like a sphere, ovoid, prolate spheroid, oblate spheroid, spheroid, cylinder, spherical polyhedron shaped, cuboctahedron, truncated tetrahedron, truncated cube, truncated octahedron, truncated dodecahedron, truncated icosahedron, truncated icosahedron, truncated cuboctahedron, icosidodecahedron, rhombicuboctahedron, rhombicosidodecahedron, rhombitruncated cuboctahedron, rhombitruncated icosidodecahedron, snub cube, snub dodecahedron, cube, dodecahedron, hexahedron, icosahedron, octahedron, and tetrahedron. The friction plug can be manufactured as part of the vascular implant as a single unitary body, or the friction plug can be attached to the vascular implant by means of welding, laser welding, ultrasonic welding, brazing, soldering, crimping, compressing, gluing, epoxying, or molding. The friction plug in one embodiment operates as the attachment between the vascular implant and the braided coil. The friction plug can be connected to at least one of the mating end, non-mating end of the vascular implant, both mating and non-mating ends, or any surface between the mating and non-mating ends of the vascular implant. The vascular implant does not have to be loaded with the friction plug into the braided coil, but for added protection, it is an option. The friction plug can be put at both ends of the vascular implant with the entire piece press fitted into the braided coil.

In the present invention, the vascular implant is delivered to the target implant site by the braided coil, but the implant and friction plug are actually ejected by the push wire. In one aspect, the push wire needs to be longer than the anticipated catheter length from the vascular access site to the implant target site. After the braided coil loaded with the friction plug and vascular implant reaches the implant target site in the patient, the push wire is inserted through the other end of the braided coil, or the proximal end and advanced along the braided coil until it contacts the friction plug loaded into the braided coil through the mating end. In another aspect, the push wire comprises of nitinol, nickel titanium, titanium alloys, brass, nickel aluminum, bronze, iron alloys, stainless steel, platinum, gold, tantalum, titanium, tungsten, metals, pseudoelastic metal alloys, shape memory alloys, polyurethane, polytetrafluoroethylene, polyvinyl alcohol, polyester, silicone, or acrylic.

In another aspect, the adhesive comprises of an acrylic adhesive, cyanoacrylate adhesive, epoxy adhesive, vinyl adhesive, silicone adhesive, rubber adhesive, urethane adhesive, methacrylic adhesive, nylon adhesive, bisphenol adhesive, diol adhesive, polyimide adhesive, polyurethane adhesive, fluoridated epoxy adhesive, and fluoridated acrylic adhesives.

In another embodiment of the present invention, the grip of the braided coil can also be increased by the apparatus further comprising a pull wire insertable into the braided coil around the friction plug. The pull wire will further increase the spring force of the filars of the braided coil on the friction plug. After the braided coil loaded with the friction plug and pull wire reaches the implant target site, the pull wire is first retracted to loosen the grip on the friction plug. The push wire would then be advanced and proceed to eject the friction plug and vascular implant from the delivery apparatus. In one aspect of the delivery apparatus utilizing the pull wire, the pull wire can either run inside the braided coil along with the push wire, or there can be an annulus on the side wall of the braided coil allowing the portion of the pull wire not in contact with the friction plug to run along the external surface of the braided coil. In another aspect, the pull wire comprises of nitinol, nickel titanium, titanium alloys, brass, nickel aluminum, bronze, iron alloys, stainless steel, platinum, gold, tantalum, titanium, tungsten, metals, psuedoelastic metal alloys, shape memory alloys, polyurethane, polytetrafluoroethylene, polyvinyl alcohol, polyester, silicone, or acrylic. In another aspect, the pull wire is also mounted on the handle to retract the pull wire after the braided coil reaches the implant target site.

In yet another embodiment of the present invention, the apparatus for delivery of a vascular implant via catheter comprising a braided coil having a mating end, a proximal end, and a lumen with a lumen diameter; a plug fitted into the mating end of the lumen wherein the plug has a distal end, a second end, and a plug diameter smaller than the lumen diameter; a vascular implant having a mating end and a non-mating end wherein the mating end of the vascular implant is connected to the distal end of the plug; a pull wire insertable into the braided coil wherein the pull wire has a wire diameter smaller than the lumen diameter; and a handle connectable to the pull wire wherein the handle can advance or retract the pull wire within the braided coil. In one aspect of the present embodiment, the plug diameter combined with the wire diameter is larger than the lumen diameter. Only when the wire diameter of the pull wire is present does the filars of the braided coil exert a spring force that grips the plug. The pull wire is inserted from the proximal end of the braided coil to extend around the plug inserted from the mating end of the braided coil. When the pull wire is not present, the plug is free to slide out the mating end of the braided coil when the braided coil is being retracted. Thus after the loaded braided coil reaches the implant target site, the pull wire will first be retracted freeing the plug inside the braided coil. The braided coil will then also be retracted allowing the deployment of the plug and vascular implant into the target implant site. In one aspect, the vascular implant is a shape memory polymer embolic foam implant for the embolization of vascular abnormalities comprising of cerebral aneurysm, intracranial aneurysms, AVMs, AVFs, peripheral vasculature, and neurovascular abnormalities.

Another aspect is that the shape of the plug in this embodiment comprises of a sphere, ovoid, prolate spheroid, oblate spheroid, spheroid, cylinder, spherical polyhedron shaped, cuboctahedron, truncated tetrahedron, truncated cube, truncated octahedron, truncated dodecahedron, truncated icosahedron, truncated icosahedron, truncated cuboctahedron, icosidodecahedron, rhombicuboctahedron, rhombicosidodecahedron, rhombitruncated cuboctahedron, rhombitruncated icosidodecahedron, snub cube, snub dodecahedron, cube, dodecahedron, hexahedron, icosahedron, octahedron, and tetrahedron. The plug can be manufactured as part of the vascular implant as a single unitary body, or the plug can be attached to the vascular implant by means of welding, laser welding, ultrasonic welding, brazing, soldering, crimping, compressing, gluing, epoxying, or molding. The plug can be connected to at least one of the mating end, non-mating end of the vascular implant, both mating and non-mating ends, or any surface between the mating and non-mating ends of the vascular implant. The vascular implant does not have to be loaded with the plug into the braided coil, but for added protection, it is an option. Plugs can be put at both ends of the vascular implant with the entire piece inside the braided coil during transport and gripped in place by the pull wire.

In another aspect, the pull wire comprises of nitinol, nickel titanium, titanium alloys, brass, nickel aluminum, bronze, iron alloys, stainless steel, platinum, gold, tantalum, titanium, tungsten, metals, psuedoelastic metal alloys, shape memory alloys, polyurethane, polytetrafluoroethylene, polyvinyl alcohol, polyester, silicone, or acrylic. In yet another aspect, the handle has an internal thread system and screw mechanism. The pull wire in another aspect can be mounted to the handle, and the handle used to advance or retract the pull wire to deploy the vascular implant. In another aspect, the handle has a knob wherein the rotation of the knob displaces the pull wire.

In another embodiment, the present invention includes a method of implanting a vascular implant via a catheter. The method includes the steps of inserting a catheter from a vascular access site to an implant target site wherein the catheter has a catheter length, providing an apparatus for delivery of a vascular implant via a catheter comprising a braided coil having a lumen, a vascular implant comprising a friction plug with a diameter that is larger than a diameter of the lumen of the braided coil and the friction plug is insertable into the braided coil, a push wire with a wire diameter smaller than the diameter of the lumen of the braided coil wherein the push wire is insertable into the braided coil, and a handle connectable to at least one of the push wire or the braided coil wherein the handle is used to advance or retract the push wire within the braided coil. Next steps by the user include prepping the braided coil if not already done so, by the manufacturer, by clipping the fixed filars on the mating end of the braided coil. This frees the set of filars of the braided coil to deflect and expand to a mating diameter larger than the original lumen diameter. User then trims the push wire so that it is longer than the anticipated catheter length and loads the vascular implant into the clipped end of the braided coil by press fitting the friction plug connected to the vascular implant into the braided coil. The freed filars of the braided coil will distend and apply a spring force that grips the friction plug of the vascular implant. The push wire and braided coil are then mounted to the handle and the user then navigates the vascular implant loaded in the braided coil through the catheter until it reaches the target treatment site. The user next advances the push wire through the braided coil until it contacts the vascular implant. Using the handle, the user then ejects the friction plug and vascular implant from the braided coil. Lastly, the user removes the braided coil and push wire from the catheter.

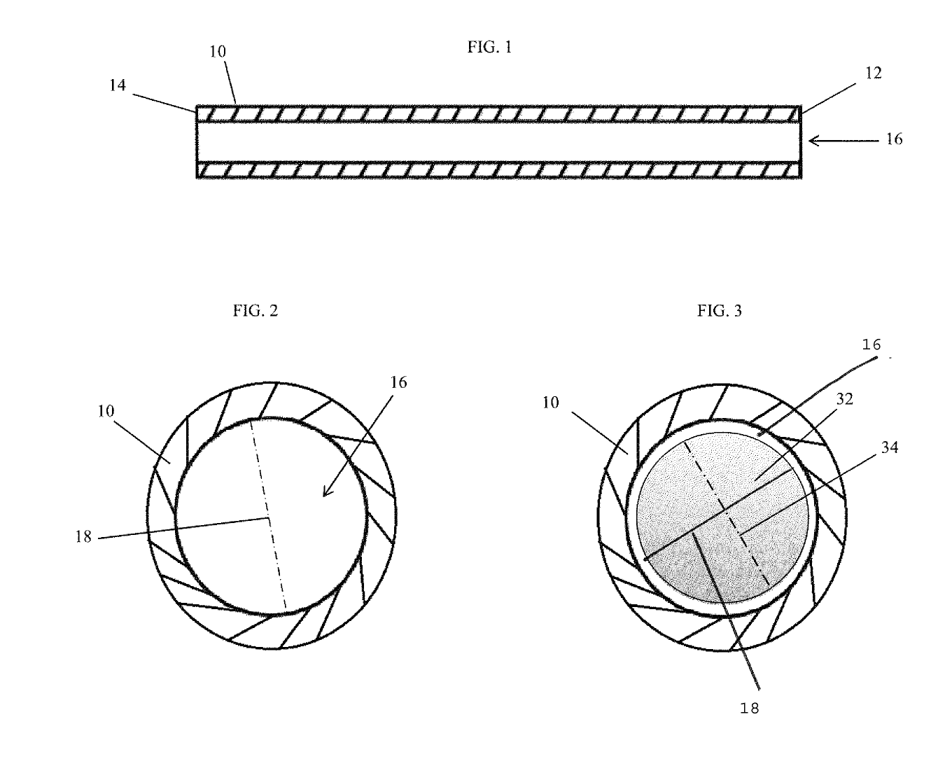

FIG. 1 is a cross-sectional view that shows the braided coil 10. In one embodiment of the present invention, the apparatus for delivery of a vascular implant via a catheter includes a braided coil 10 with a lumen 16, a distal end 12, and a proximal end 14.

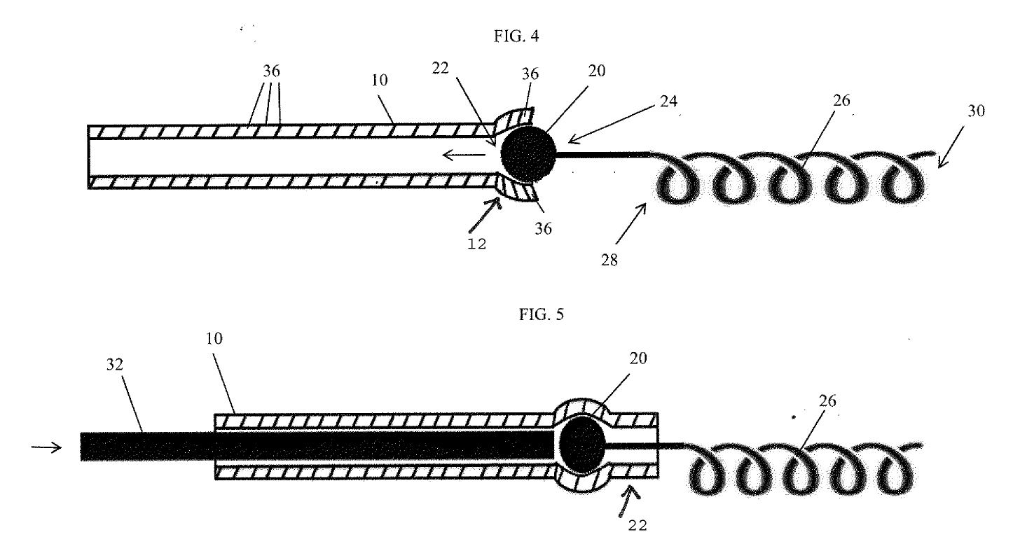

FIG. 2 is a cross-sectional view that shows the braided coil 10. FIG. 2 shows the lumen 16 has a lumen diameter 18, and that the apparatus also includes a push wire 32 (see FIG. 3) that is insertable into braided coil 10. FIG. 3 shows that the push wire 32 has a wire diameter 34 that is smaller than the lumen diameter 18 allowing it to easily maneuver inside braided coil 10.

FIG. 3 is a cross-sectional view that shows the braided coil 10 and the guide wire 32. FIG. 3 shows the lumen 16 has a lumen diameter 18, and that the apparatus also includes a push wire 32 that is insertable into braided coil 10. FIG. 3 also shows that the push wire 32 has a wire diameter 34 that is smaller than the lumen diameter 18 allowing it to easily maneuver inside braided coil 10.

FIG. 4 is a cross-sectional view that shows the friction plug 20 being loaded into braided coil 10. FIG. 4 also shows how a vascular implant 26 is loaded into the braided coil 10 to be delivered to target implant site. Friction plug 20 has a proximal end 22 and second end 24. The second end 24 of friction plug 20 is connected to vascular implant 26. Friction plug 20 is frictionally fitted into braided coil 10 with proximal end 22 going in first. Friction plug 20 is connected to the mating end 28 of vascular implant 26. In various embodiments, friction plug 20 can be connected not only to mating end 28, but also non-mating end 30 of vascular implant 26, or both. There can be more than one friction plug 20 connected to vascular implant 26 and press fitted into braided coil 10 for delivery. To load friction plug 20 into braided coil 10, the set of filars 36 in distal end 12 of braided coil 10 must be clipped or unfixed to allow the set of filars 36 to be free to deflect and expand. Friction plug 20 has a plug diameter larger than the lumen diameter of braided coil 10, and the set of filars 36 must expand and friction plug 20 press fitted into braided coil 10. The set of filars 36 will distend and apply a spring force on friction plug 20 that grips friction plug 20 during delivery.

FIG. 5 is a cross sectional view that shows the friction plug 20 loaded into braided coil 10 and including push wire 32. FIG. 5 also shows one embodiment of the present invention delivery apparatus in use. Braided coil 10 is loaded with friction plug 20 and vascular implant 26. The loaded braided coil is then navigated to the implant target site. After reaching the implant target site, push wire 32 is brought in contact with the proximal end 22 of friction plug 20. After the braided coil 10 loaded with friction plug 20 and vascular implant 26 reaches the implant target site, push wire 32 is advanced in braided coil 10 until it reaches friction plug 20. User will prepare to eject the vascular implant 26 into the implant target site by ejecting the friction plug 20 from the braided coil using push wire 32.

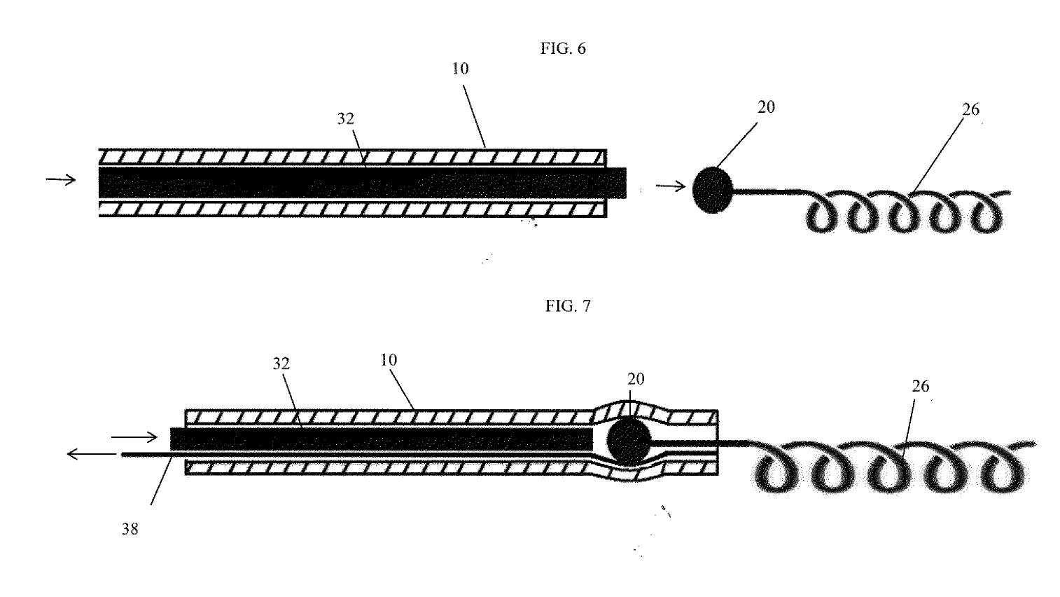

FIG. 6 is a cross-sectional view that shows the delivery apparatus releasing vascular implant 26. FIG. 6 also shows the present invention delivering a vascular implant 26 by ejecting friction plug 20 from braided coil 10 using push wire 32. Push wire 32 ejects friction plug 20 by using a handle with a screw mechanism and internal threads that both push wire 32 and braided coil 10 are mounted to.

FIG. 7 is a cross sectional view of another embodiment of the delivery apparatus releasing vascular implant 26 that show the friction plug 20 loaded into braided coil 10 and including push wire 32. FIG. 7 shows another embodiment of the present invention wherein the delivery apparatus further includes a pull wire 38 to increase grip strength on the friction plug 20. After the braided coil 10 loaded with friction plug 20 reaches the target implant site, the user retracts pull wire 38 and advances push wire 32 to eject friction plug 20.

FIG. 8 is a cross sectional view of another embodiment of the delivery apparatus releasing vascular implant 26 that shows the friction plug 20 loaded into braided coil 10 and including the push wire 32. FIG. 8 also shows another embodiment of the present invention wherein an annulus 40 in the sidewall of the braided coil 10 allows the pull wire 38 to be retracted along the external surface of the braided coil 10.

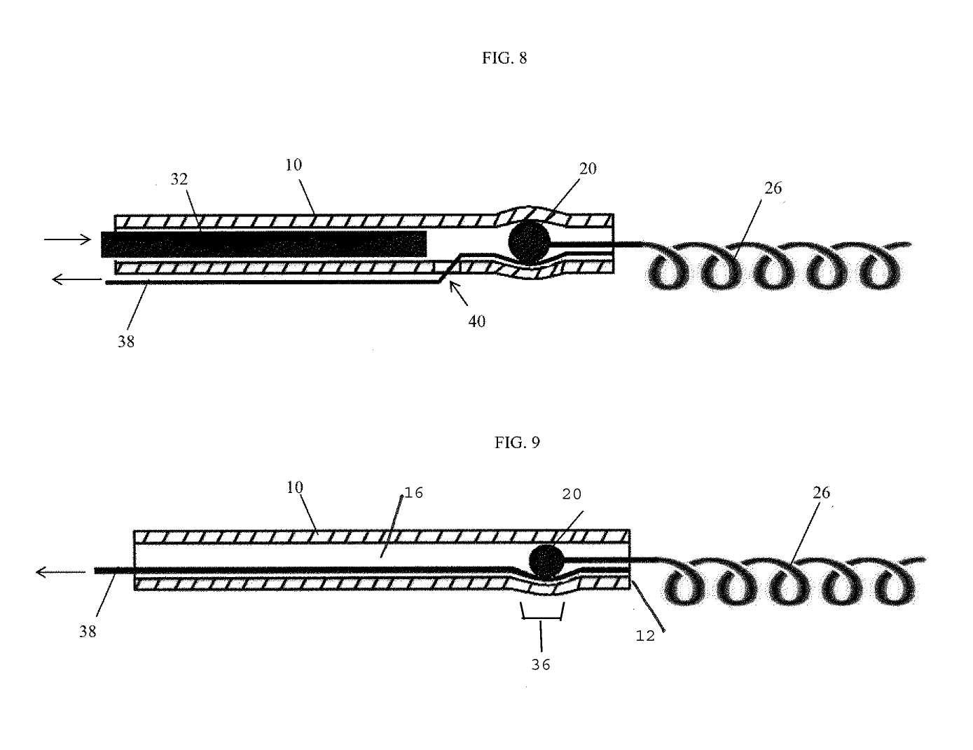

FIG. 9 is a cross sectional view of another embodiment of the delivery apparatus releasing vascular implant 26 that shows the friction plug 20 loaded into braided coil 10 and including the pull wire 38. FIG. 9 also shows yet another embodiment of the present invention wherein the plug 20 is instead sized to fit inside the lumen 16 of braided coil 10. The plug 20 only deflects the set of filars 36 of braided coil 10 when pull wire 38 is in place. This embodiment ejects plug 20 by first retracting pull wire 38, and then retracting the braided coil 10. When braided coil 10 is being retracted, the plug 20 exits the distal end 12 of braided coil 10 and is deployed at the implant target site. In another embodiment the braided coil (not shown) may include a device for preventing filar deflection via placing a solid band (not shown) around the braided coil (not shown) or welding the filars 36 (not shown) together on the proximal and distal sides of the friction plug (not shown).

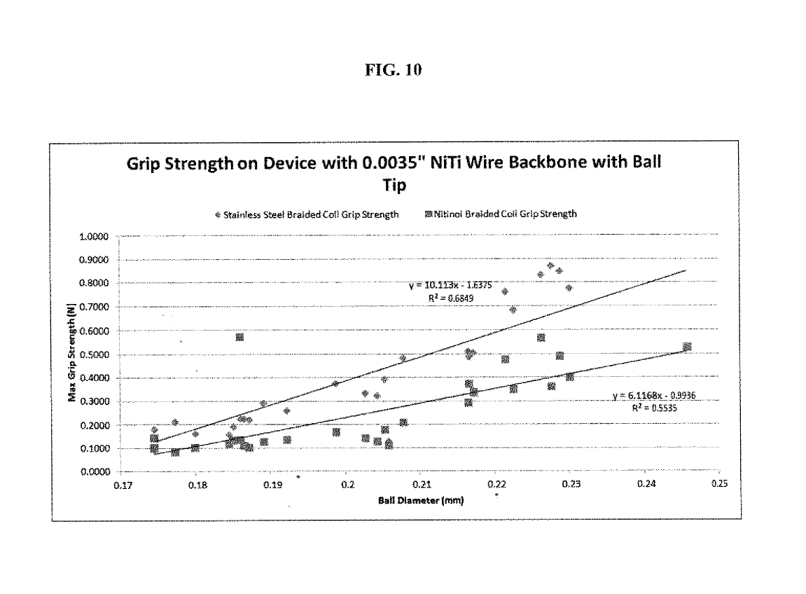

FIG. 10 is a plot that shows the grip strength based on the diameter of the friction plug and the material of the braided coil catheter (stainless steel and nitinol), in which the ball diameter and material of the braided coil is 0.006″ I.D. and 0.012″ O.D. The device had a 0.0035″ NiTi wire backbone with a spherical friction plug.

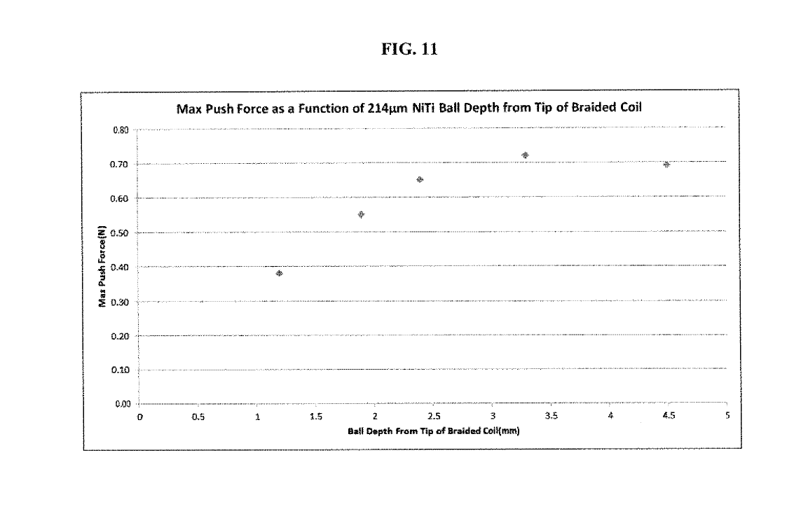

FIG. 11 is a plot that shows the max push force required to eject the friction plug from the braided coil as a function of the depth of the nitinol friction plug from the tip of the braided coil. The force required to push a device with a 214 μm diameter spherical friction plug from a stainless steel braided coil with 0.006″ I.D. and 0.012″ O.D.

In another aspect of the present invention, the push wire, the braided coil, or both are mounted to the handle. After the push wire contacts the friction plug, the friction plug is ejected by the push wire using the handle. In another aspect, the handle has an internal thread system and screw mechanism that can advance or retract the push wire. In yet another aspect, the handle also has a knob wherein the rotation of the knob displaces the push wire.

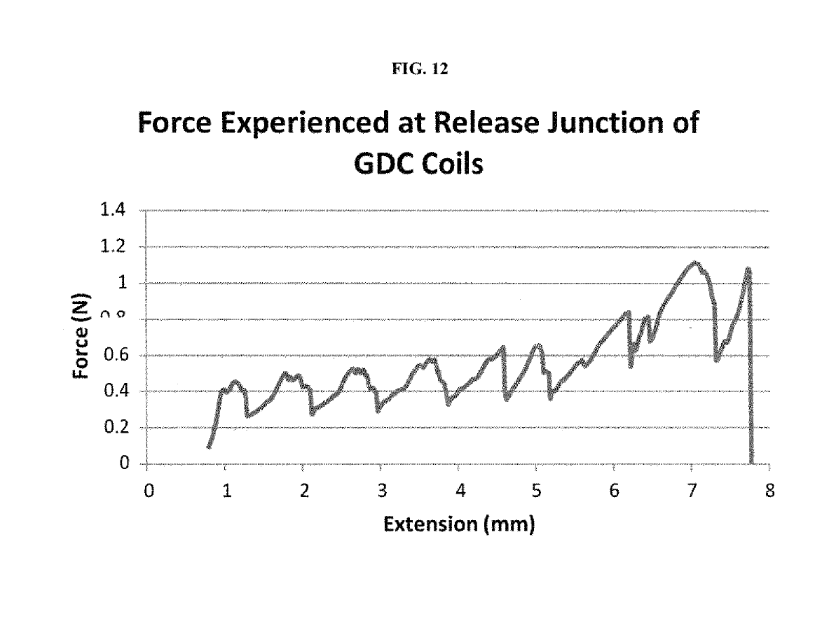

FIG. 12 is a graph that shows a comparison between the vascular implant delivery system of the instant invention and a guglielmi detachable coil (GDC) delivery system. The graph shows the force experienced at the release junction of the GDC coils.

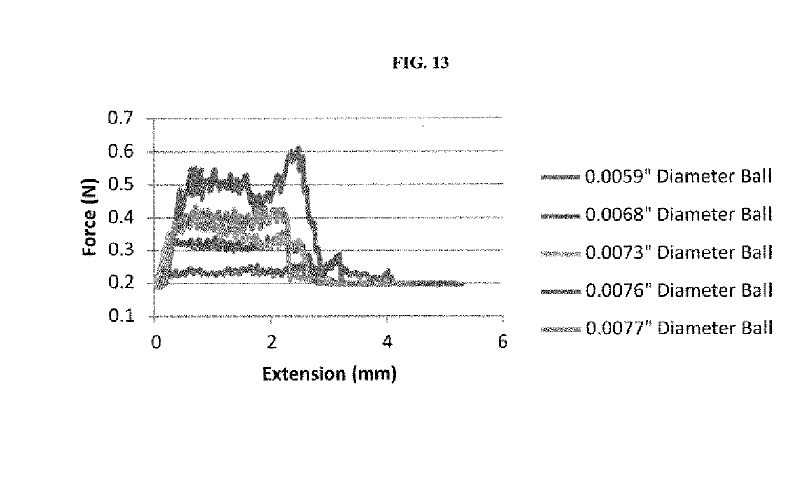

FIG. 13 is a graph that shows the grip strength of stainless steel braided coils with a 0.002″ diameter NiTi pull wire and restrained filar movement.

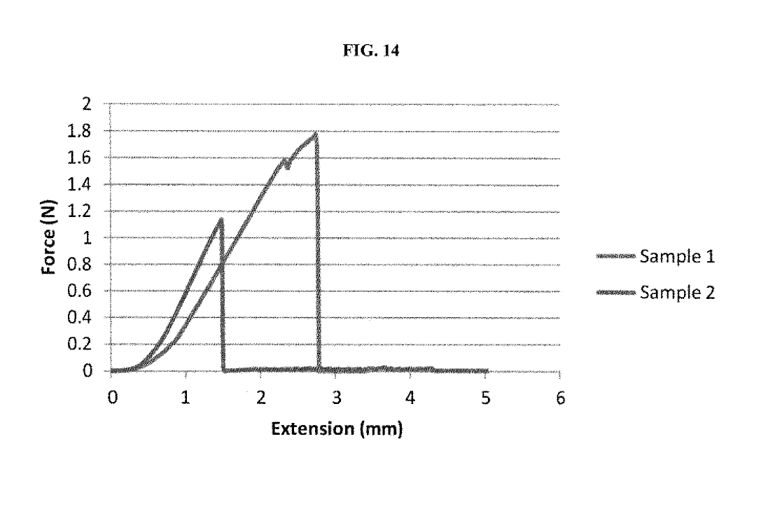

FIG. 14 is a graph that shows the grip strength of stainless steel braided coils with a 0.003″ diameter NiTi pull wire and restrained filar movement.

In another embodiment of the present invention, the grip of the braided coil on the friction plug can be increased by restricting the deflection of the set of filars making up the braided coil with an adhesive or through welding the individual filars together.

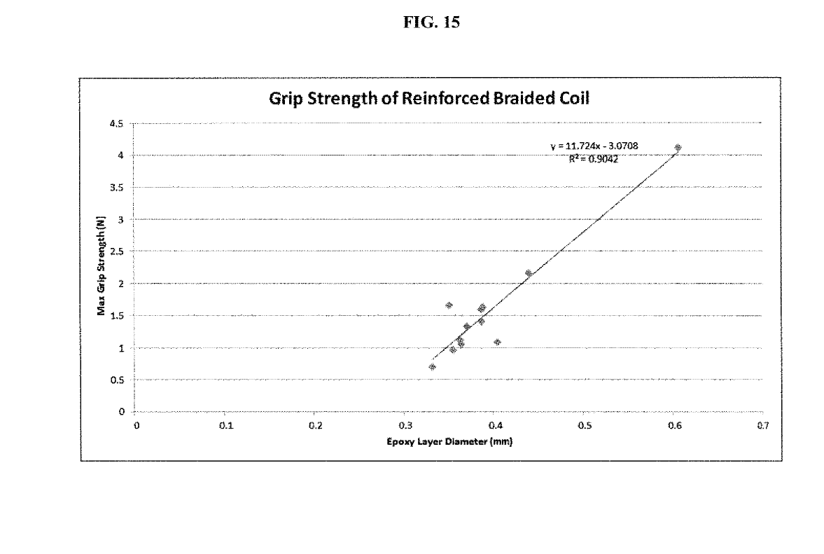

FIG. 15 is a graph that shows the increased grip strength achieved based on using epoxy to restrict filar deflection on the braided coil.

The present invention provides a braided coil catheter to delivery of a vascular implant through a catheter including a braided coil adapted to at least partially retain a vascular implant comprising a braided coil distal tip, a braided coil proximal end connected by a braided coil body having a lumen with a lumen diameter; a friction plug comprising a proximal friction plug end, a distal friction plug end connected by a friction plug body with a friction plug diameter greater than the lumen diameter to frictionally fit into the lumen of the braided coil distal tip; a vascular implant having an implant mating end, an implant non-mating end connected by an implant body wherein the implant mating end is connected to the distal friction plug end; and a pusher catheter inserted into the lumen and to contact the proximal friction plug end wherein the pusher catheter can force the friction plug from the lumen.

The apparatus may include a retaining wire positioned in the lumen to frictionally retain the friction plug. The apparatus may also include a handle connected to the retaining wire, the pusher catheter, the braided coil, or a combination thereof to move the retaining wire or the pusher catheter within the braided coil. The braided coil may include a set of filars connected to the braided coil distal tip to deflect and expand to a mating diameter larger than the lumen diameter, wherein the set of filars distends and applies a spring force that grips the friction plug when frictionally fitted into the lumen. The lumen diameter may be between 0.001 and 0.010 and more specifically about 0.005, 0.0055, 0.006, 0.0065, or 0.007 inches. The braided coil has an outer diameter which may be between 0.005 and 0.020 and about 0.01, 0.011, 0.012, 0.013, 0.014, or 0.015 inches. The braided coil at least partially made of nitinol, nickel titanium, titanium alloys, brass, nickel aluminum, bronze, iron alloys, stainless steel, platinum, gold, tantalum, titanium, tungsten, metals, psuedoelastic metal alloys, shape memory alloys, polyurethane, polytetrafluoroethylene, polyvinyl alcohol, polyester, silicone, or acrylic. The friction plug may be a sphere, ovoid, prolate spheroid, oblate spheroid, spheroid, cylinder, spherical polyhedron shaped, cuboctahedron, truncated tetrahedron, truncated cube, truncated octahedron, truncated dodecahedron, truncated icosahedron, truncated icosahedron, truncated cuboctahedron, icosidodecahedron, rhombicuboctahedron, rhombicosidodecahedron, rhombitruncated cuboctahedron, rhombitruncated icosidodecahedron, snub cube, snub dodecahedron, cube, dodecahedron, hexahedron, icosahedron, octahedron, or tetrahedron. The friction plug may form a single unitary body with the vascular implant or be connected to the vascular implant by welding, laser welding, ultrasonic welding, brazing, soldering, crimping, compressing, gluing, epoxying, or molding. The friction plug at least partially made of nitinol, nickel titanium, titanium alloys, brass, nickel aluminum, bronze, iron alloys, stainless steel, platinum, gold, tantalum, titanium, tungsten, metals, psuedoelastic metal alloys, shape memory alloys, polyurethane, polytetrafluoroethylene, polyvinyl alcohol, polyester, silicone, acrylic or a combination thereof. The retaining wire may be made of nitinol, nickel titanium, titanium alloys, brass, nickel aluminum, bronze, iron alloys, stainless steel, platinum, gold, tantalum, titanium, tungsten, metals, psuedoelastic metal alloys, shape memory alloys, polyurethane, polytetrafluoroethylene, polyvinyl alcohol, polyester, silicone, or acrylic. The vascular implant may be a shape memory polymer embolic foam implant for the embolization of vascular abnormalities comprising of cerebral aneurysm, intracranial aneurysms, AVMs, AVFs, peripheral vasculature, and neurovascular abnormalities.

The retaining wire may be removably positioned at least partially between the friction plug and the braided coil. The handle further may include a screw mechanism with an internal thread system to move the retaining wire, the push wire or both. An adhesive may be applied to the lumen to modify the grip of the friction plug wherein the adhesive is selected from an acrylic adhesive, a cyanoacrylate adhesive, an epoxy adhesive, a vinyl adhesive, a silicone adhesive, a rubber adhesive, an urethane adhesive, a methacrylic adhesive, a nylon adhesive, a bisphenol adhesive, a diol adhesive, a polyimide adhesive, a polyurethane adhesive, a fluoridated epoxy adhesive, and a fluoridated acrylic adhesives to achieve greater grip strength on the frictional plug.

The present invention also includes a method of implanting a vascular implant by moving a catheter from a vascular access site to an implant target site; providing an apparatus for delivery of a vascular implant comprising a braided coil comprising a braided coil distal end, a braided coil proximal end connected by a braided coil body having a lumen with a lumen diameter; a friction plug comprising a proximal friction plug end, a distal friction plug end connected by a friction plug body with a friction plug diameter greater than the lumen diameter frictionally fitted into the lumen of the braided coil distal end; providing a vascular implant having an implant mating end, an implant non-mating end connected by a an implant body wherein the implant mating end is connected to the proximal friction plug end; loading the vascular implant into the braided coil by frictionally fitting the friction plug into the braided coil to form a loaded braided coil; navigating the loaded braided coil through the catheter to reach the implant target site; advancing the wire through the braided coil until the wire contacts the friction plug; ejecting the friction plug from the braided coil to deploy the vascular implant into the implant target site; and removing the braided coil and wire from the catheter.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An apparatus for delivery of a vascular implant through a catheter, comprising:
a braided coil adapted to at least partially retain a vascular implant comprising a braided coil distal tip, a braided coil proximal end connected by a braided coil body having a lumen with a lumen diameter;
a friction plug comprising a proximal friction plug end, a distal friction plug end connected by a friction plug body with a friction plug diameter greater than the lumen diameter to frictionally fit into the lumen of the braided coil distal tip;
a vascular implant having an implant mating end, an implant non-mating end connected by an implant body wherein the implant mating end is connected to the distal friction plug end; and
a pusher catheter inserted into the lumen and to contact the proximal friction plug end wherein the pusher catheter can force the friction plug from the lumen.

2. The apparatus of claim 1, further comprising a retaining wire positioned in the lumen to frictionally retain the friction plug.

3. The apparatus of claim 2, further comprising a handle connected to the retaining wire, the pusher catheter, the braided coil, or a combination thereof to move the retaining wire or the pusher catheter within the braided coil.

4. The apparatus of claim 1, wherein the braided coil comprises a set of filars connected to the braided coil distal tip to deflect and expand to a mating diameter larger than the lumen diameter.

5. The apparatus of claim 4, wherein the set of filars distends and applies a spring force that grips the friction plug when frictionally fitted into the lumen.

6. The apparatus of claim 1, wherein the lumen diameter is about 0.005, 0.0055, 0.006, 0.0065, or 0.007 inches.

7. The apparatus of claim 1, wherein the braided coil has an outer diameter about 0.01, 0.011, 0.012, 0.013, 0.014, 0.015, 0.016, 0.017, or 0.018 inches.

8. The apparatus of claim 1, wherein the braided coil at least partially comprises nitinol, nickel titanium, titanium alloys, brass, nickel aluminum, bronze, iron alloys, stainless steel, platinum, gold, tantalum, titanium, tungsten, metals, psuedoelastic metal alloys, shape memory alloys, polyurethane, polytetrafluoroethylene, polyvinyl alcohol, polyester, silicone, or acrylic.

9. The apparatus of claim 1, wherein the friction plug is a sphere, ovoid, prolate spheroid, oblate spheroid, spheroid, cylinder, spherical polyhedron shaped, cuboctahedron, truncated tetrahedron, truncated cube, truncated octahedron, truncated dodecahedron, truncated icosahedron, truncated icosahedron, truncated cuboctahedron, icosidodecahedron, rhombicuboctahedron, rhombicosidodecahedron, rhombitruncated cuboctahedron, rhombitruncated icosidodecahedron, snub cube, snub dodecahedron, cube, dodecahedron, hexahedron, icosahedron, octahedron, or tetrahedron.

10. The apparatus of claim 1, wherein the friction plug forms a single unitary body with the vascular implant.

11. The apparatus of claim 1, wherein the friction plug is connected to the vascular implant by welding, laser welding, ultrasonic welding, brazing, soldering, crimping, compressing, gluing, epoxying, or molding.

12. The apparatus of claim 1, wherein the friction plug at least partially comprises nitinol, nickel titanium, titanium alloys, brass, nickel aluminum, bronze, iron alloys, stainless steel, platinum, gold, tantalum, titanium, tungsten, metals, psuedoelastic metal alloys, shape memory alloys, polyurethane, polytetrafluoroethylene, polyvinyl alcohol, polyester, silicone, acrylic or a combination thereof.

13. The apparatus of claim 1, wherein the retaining wire comprises of nitinol, nickel titanium, titanium alloys, brass, nickel aluminum, bronze, iron alloys, stainless steel, platinum, gold, tantalum, titanium, tungsten, metals, psuedoelastic metal alloys, shape memory alloys, polyurethane, polytetrafluoroethylene, polyvinyl alcohol, polyester, silicone, or acrylic.

14. The apparatus of claim 1, wherein the vascular implant is a shape memory polymer embolic foam implant for the embolization of vascular abnormalities comprising of cerebral aneurysm, intracranial aneurysms, AVMs, AVFs, peripheral vasculature, and neurovascular abnormalities.

15. The apparatus of claim 1, wherein the retaining wire is removably positioned at least partially between the friction plug and the braided coil.

16. The apparatus of claim 1, wherein the handle further comprises a screw mechanism with an internal thread system to move the retaining wire, the pusher catheter or both.

17. The apparatus of claim 1, further comprising a restriction in the braided coil to at least partially frictionally fit the friction plug.

18. The apparatus of claim 1, further comprising an adhesive applied to the lumen to modify the grip of the friction plug wherein the adhesive is selected from an acrylic adhesive, a cyanoacrylate adhesive, an epoxy adhesive, a vinyl adhesive, a silicone adhesive, a rubber adhesive, an urethane adhesive, a methacrylic adhesive, a nylon adhesive, a bisphenol adhesive, a diol adhesive, a polyimide adhesive, a polyurethane adhesive, a fluoridated epoxy adhesive, and a fluoridated acrylic adhesives to achieve greater grip strength on the frictional plug.

19. A method of implanting a vascular implant comprising the steps of:
moving a catheter from a vascular access site to an implant target site;
providing an apparatus for delivery of a vascular implant comprising
a braided coil comprising a braided coil distal end, a braided coil proximal end connected by a braided coil body having a lumen with a lumen diameter; a friction plug comprising a proximal friction plug end, a distal friction plug end connected by a friction plug body with a friction plug diameter greater than the lumen diameter frictionally fitted into the lumen of the braided coil distal end;
providing a vascular implant having an implant mating end, an implant non-mating end connected by a an implant body wherein the implant mating end is connected to the proximal friction plug end;
loading the vascular implant into the braided coil by frictionally fitting the friction plug into the braided coil to form a loaded braided coil;
navigating the loaded braided coil through the catheter to reach the implant target site;
advancing the pusher catheter through the braided coil until the wire contacts the friction plug;
ejecting the friction plug from the braided coil to deploy the vascular implant into the implant target site; and
removing the braided coil and pusher catheter from the catheter.

20. The method of claim 19, further comprising the step of clipping the mating end of the braided coil to insert the friction plug.

21. The method of claim 19, wherein the braided coil distends and applies a spring force to the friction plug.

22. The method of claim 19, wherein the retaining wire, the braided coil, or both are mounted to the handle.

23. The method of claim 19 wherein the handle advances the pusher catheter to eject the friction plug, the vascular implant, or both from the braided coil.
